(19) United States
(12) Patent Application Publication
(10) Pub. No.: US 2025/0156604 A1
(43) Pub. Date: May 15, 2025

(54) FINITE ELEMENT SIMULATION TECHNOLOGY-BASED ATMOSPHERIC-CORROSION PREDICTION METHOD FOR AIR-CONDITIONER HEAT EXCHANGER

(71) Applicant: CHINA NATIONAL ELECTRIC APPARATUS RESEARCH INSTITUTE CO., LTD., Guangdong (CN)

(72) Inventors: Yonggao FU, Guangdong (CN); Jun WANG, Guangdong (CN); Li QI, Guangdong (CN); Miaoran LIU, Guangdong (CN); Huang PENG, Guangdong (CN); Yang WANG, Guangdong (CN)

(73) Assignee: CHINA NATIONAL ELECTRIC APPARATUS RESEARCH INSTITUTE CO., LTD., Guangdong (CN)

(21) Appl. No.: 19/021,329

(22) Filed: Jan. 15, 2025

Related U.S. Application Data
(63) Continuation of application No. PCT/CN2023/107014, filed on Jul. 12, 2023.

(30) Foreign Application Priority Data
Jul. 28, 2022 (CN) 202210901946.X

(51) Int. Cl. G06F 30/23; G01N 17/00; G06F 113/14

(52) U.S. Cl. CPC G06F 30/23; G01N 17/006; G06F 2113/14

(57) ABSTRACT

A finite element simulation technology-based atmospheric-corrosion prediction method for an air-conditioner heat exchanger is provided. In the method, a material corrosion prediction model for the material of a heat conduction pipe of an air-conditioner heat exchanger to be tested and the material of heat dissipation fins of said air-conditioner heat exchanger is debugged by means of step S1 and step S2; an assembly corrosion prediction model is then optimized by means of step S3 and step S4; and when a multi-physics field in an air-conditioner serving environment is comprehensively simulated and coupled by using a working condition environment field, the atmospheric corrosion of said air-conditioner heat exchanger after operating in an outdoor serving environment to be tested under a working condition for any preset duration is finally predicted by means of step S5 and step S6, such that an atmospheric-corrosion prediction result is obtained.

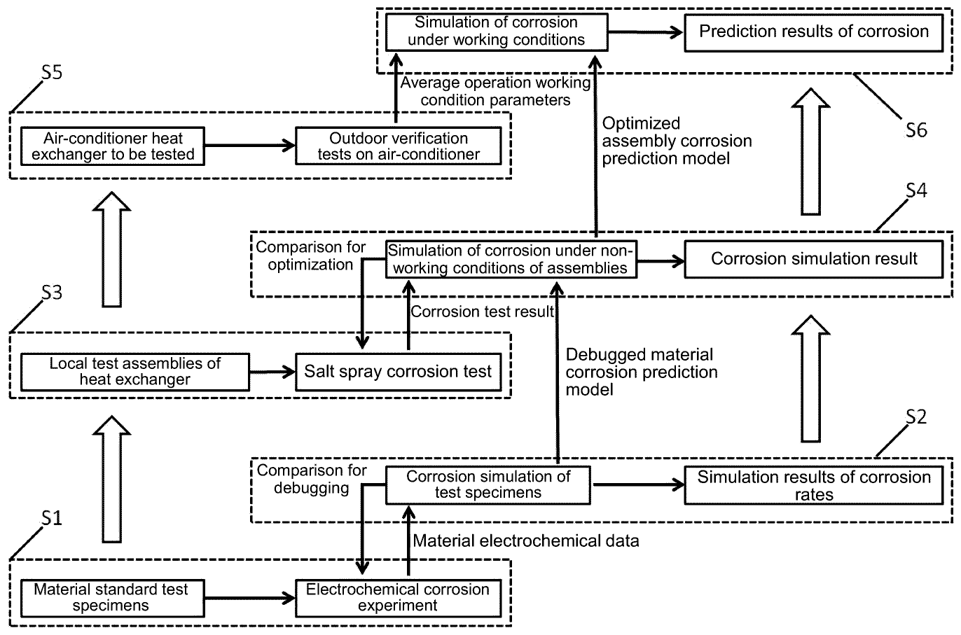

### CROSS REFERENCE TO RELATED APPLICATIONS

This application is a continuation of international application of PCT application serial no. PCT/CN2023/107014, filed on Jul. 12, 2023, which claims the priority benefit of China application no. 202210901946.X filed on Jul. 28, 2022. The entirety of each of the above mentioned patent applications is hereby incorporated by reference herein and made a part of this specification.

### TECHNICAL FIELD

The present disclosure relates to an atmospheric-corrosion prediction method for electrical equipment, specifically a finite element simulation technology-based atmospheric-corrosion prediction method for air-conditioner heat exchanger.

### DESCRIPTION OF RELATED ART

In accordance with the national strategic planning for high-quality development of the

marine economy, the construction of various maritime facilities is being expedited, including the gradual enhancement of naval and reef military support facilities in coastal and island areas. The majority of China's maritime regions are subject to harsh environmental conditions characterized by high temperature, high humidity, and high salt fog concentration, collectively known as the “three highs.” These conditions exert a more severe corrosive impact on the metallic components of electrical products compared to inland areas. Concurrently, air-conditioner systems are indispensable civilian electrical appliances in maritime regions and constitute a crucial element of military installations. The heat exchanger, being one of the most critical components of an air-conditioner system, directly influences the heat transfer performance of the unit through its structural integrity. Consequently, it is imperative to investigate the atmospheric-corrosion behavior of air-conditioner heat exchangers in marine environments. The utilization of finite element simulation technology to predict the corrosion sites, corrosion rates, and the impact of corrosion on heat transfer performance of air-conditioner heat exchangers is warranted. This approach will provide guidance for corrosion-resistant design and external corrosion protection measures for air-conditioner heat exchangers, thereby reducing the necessity for extensive experimental work and associated time and financial expenditures.

The essence of atmospheric-corrosion of metals is electrochemical-corrosion under thin liquid films of varying thicknesses. The variation in the thickness of these thin liquid films directly affects the mass transfer process of electrode reactions, thereby significantly impacting the atmospheric-corrosion rates of metal components. According to current theoretical research, the thickness of thin liquid films is primarily influenced by, for example, temperature, humidity, and salt deposition. Among these factors, temperature conditions are not only affected by ambient temperature but also by the surface temperature variations of air-conditioner heat exchangers during operation. Therefore, to accurately predict the atmospheric-corrosion of air-conditioner heat exchangers, it is necessary to comprehensively consider and couple multiple physical fields present in the service environment of air conditioners, in order to more precisely simulate the atmospheric-corrosion behavior of air-conditioner heat exchangers.

### SUMMARY

The technical problem to be solved by the present disclosure is: to provide a finite element simulation technology-based atmospheric-corrosion prediction method for air-conditioner heat exchanger.

To solve the above technical problem, the present disclosure adopts the following technical solution:

A finite element simulation technology-based atmospheric-corrosion prediction method for air-conditioner heat exchanger, characterized in that the method includes:

In step S1, an experimental result of the corrosion rates of the material standard test

specimens of a heat conduction pipe and the material standard test specimens of a heat dissipation fin is obtained through an electrochemical-corrosion experiment; wherein, the material standard test specimens of the heat conduction pipe and the material standard test specimens of the heat dissipation fin are made from the material of the heat conduction pipe and the material of the heat dissipation fin of an air-conditioner heat exchanger to be tested.

In step S2, based on the experimental parameters and result of step S1, simulation results of corrosion rates of the material standard test specimens of the heat conduction pipe and the material standard test specimens of the heat dissipation fin are obtained through simulation software; and, debugging is performed to obtain a debugged material corrosion prediction model that meets the required accuracy according to the comparison between the simulation results of the corrosion rates and the experimental result of the corrosion rates mentioned in step S1.

In step S3, salt spray corrosion tests are performed on local test assemblies of the heat exchanger under non-working conditions to obtain the corrosion test result, which involves a corroded area, the morphology and type of corrosion and the size of the corrosion, for the local test assemblies of the heat exchanger.

In step S4, based on the test parameters and result of step S3, the corrosion simulation results involving the corroded area, the morphology and type of corrosion and the size of the corrosion for the local test assemblies of the heat exchanger are obtained through simulation software; and, optimization is performed to obtain the optimized assembly corrosion prediction model that meets the required accuracy according to the comparison between the corrosion simulation results and the corrosion test result mentioned in step S3.

In step S5, an air-conditioner with an outdoor unit including the air-conditioner heat exchanger to be tested is adopted, and the outdoor unit is mounted in the outdoor serving environment to be tested so as to conduct outdoor verification tests on the air-conditioner under working conditions, thus obtaining average working condition parameters of the air-conditioner that may affect the corrosion behavior of the air-conditioner heat exchanger to be tested during the verification test duration.

In step S6, based on the average working condition parameters mentioned in step S5 and the optimized assembly corrosion prediction model mentioned in step S4, the atmospheric-corrosion prediction result for the air-conditioner heat exchanger to be tested after working for any preset duration in the outdoor serving environment to be tested under working conditions is obtained through simulation software, wherein the atmospheric-corrosion prediction result involves the corroded area, the morphology and type of corrosion and the size of the corrosion.

The specific process of step S1 includes:

In step S1-1, under preset experimental environmental conditions, the electrochemical-corrosion experiment is conducted on the material standard test specimens of the heat conduction pipe and the material standard test specimens of the heat dissipation fin respectively to obtain the material electrochemical data during corrosion of the material of the heat conduction pipe and the material of the heat dissipation fin; wherein, the experimental environmental conditions include temperature, relative humidity, and salt concentration. Moreover, the temperature value, relative humidity value, and salt concentration value of the experimental environmental conditions are set to the average atmospheric temperature, average relative humidity, and average salt spray particle concentration in the air calculated in step S5-4 respectively. The material electrochemical data include anodic exchange current density, anodic Tafel slope, cathode exchange current density, and cathode Tafel slope.

Generally, the material of heat conduction pipe and material of heat dissipation fin are copper and aluminum respectively, but the present disclosure does not exclude cases where the heat conduction pipes and heat dissipation fins of the air-conditioner heat exchanger to be tested are made of other materials.

In step S1-2, based on the material electrochemical data obtained in step S1-1, calculation is performed to obtain the experimental result of corrosion rates of the material standard test specimens of heat conduction pipe and the material standard test specimens of heat dissipation fin under the experimental environmental conditions.

Preferably: in step S1-1, both the material standard test specimens of heat conduction pipe and the material standard test specimens of heat dissipation fin are cuboids with length, width, and thickness of 10 mm, 10 mm, and 3 mm respectively.

The specific process of step S2 includes:

In step S2-1, digitalized geometric models of the material standard test specimens of heat conduction pipe and the material standard test specimens of heat dissipation fin are constructed respectively, and the digitalized geometric models of the two material standard test specimens are introduced into simulation software; wherein, the digitalized geometric models of the material standard test specimens may be constructed through three-dimensional modeling software such as Solidworks, Auto CAD, etc.; the simulation software may be selected from simulation software with pre-set atmospheric-corrosion simulation models such as Ansys, Comsol, etc.

In step S2-2, the pre-set atmospheric-corrosion simulation model in the simulation software is adopted, and the material electrochemical data during corrosion of the material of heat conduction pipe and the material of heat dissipation fin obtained in step S1 are used as boundary conditions, so as to construct a material corrosion prediction model based on shell current distribution.

In step S2-3, in the simulation software, the material corrosion prediction model is adopted to conduct corrosion simulation calculations on the digitalized geometric models of the two material standard test specimens, thereby obtaining the simulation results of corrosion rates the material standard test specimens of heat conduction pipe and the material standard test specimens of heat dissipation fin under the same environmental conditions as the experimental environmental conditions described in step S1-1.

In step S2-4, if the error between the simulation results of corrosion rates of the material standard test specimens of heat conduction pipe and the material standard test specimens of heat dissipation fin and the experimental result of corrosion rates obtained in step S1 is greater than the preset corrosion rate error threshold, it means that the current material corrosion prediction model has not met the accuracy requirements. Then, after debugging the parameters of the material corrosion prediction model (generally the debugging method is to modify the model parameters to different empirical values or change the model parameters progressively according to some rules), steps S2-3 and S2-4 are repeated until the error between the simulation results of corrosion rates of the material standard test specimens of heat conduction pipe and the material standard test specimens of heat dissipation fin and the experimental result of corrosion rates obtained in step S1 is less than the corrosion rate error threshold, which means that the current material corrosion prediction model has met the accuracy requirements, and the current material corrosion prediction model is then used as the debugged material corrosion prediction model. The value range of the corrosion rate error threshold is between 5% and 10%.

The specific process of step S3 includes:

In step S3-1, a portion of the heat conduction pipes and a portion of the heat dissipation fins are cut from the air-conditioner heat exchanger to be tested through a metal cutting machine to serve as local test assemblies of the heat exchanger. Moreover, the contact method between heat conduction pipes and heat dissipation fins in the local test assemblies of the heat exchanger is consistent with the contact method between the heat conduction pipes and the heat dissipation fins in the air-conditioner heat exchanger to be tested, thus ensuring good contact between the material of heat conduction pipes and the material of heat dissipation fins.

In step S3-2, the local test assemblies of the heat exchanger are placed in a salt spray test chamber, and constant test environmental conditions are set for the salt spray test chamber. After the placement time of the local test assemblies of the heat exchanger in the salt spray test chamber reaches the preset salt spray corrosion test duration, the local test assemblies of the heat exchanger are removed for appearance observation and calibration experiment measurement, thus obtaining the corrosion test result involving the corroded area, the morphology and type of corrosion and the size of the corrosion for the local test assemblies of the heat exchanger; wherein, the test environmental conditions are the same as the experimental environmental conditions described in step S1-1. The salt concentration is realized by spraying salt solution with a mass fraction into the salt spray test chamber. The corroded area is generally divided into three types of corroded areas: corrosion occurring on heat conduction pipes, corrosion occurring on heat dissipation fins, and corrosion occurring simultaneously on heat conduction pipes and heat dissipation fins.

The specific process of step S4 includes:

In step S4-1, the digitalized geometric model of the local test assemblies of the heat exchanger is constructed, and the digitalized geometric model of the local test assemblies is introduced into simulation software. The heat conduction pipe portion and heat dissipation fin portion in the digitalized geometric model of the local test assemblies are set with corresponding material properties, that is: the heat conduction pipe portion and heat dissipation fin portion in the digitalized geometric model of the local test assemblies are respectively set as the material of heat conduction pipe and the material of heat dissipation fin described in step S1-1 respectively. The digitalized geometric model of the local test assemblies may be constructed through three- dimensional modeling software such as Solidworks, Auto CAD, etc.; the simulation software may be selected from simulation software such as Ansys, Comsol, etc.

In step S4-2, the debugged material corrosion prediction model described in step S2 is adopted in the simulation software, and the test environmental conditions described in step S3-2 and the material electrochemical data of the material of heat conduction pipe and the material of heat dissipation fin during corrosion obtained in step SI are used as boundary conditions to construct an assembly corrosion prediction model based on shell current distribution.

In step S4-3, the assembly corrosion prediction model in the simulation software is adopted to conduct corrosion simulation calculation on the digitalized geometric model of local test assemblies according to the salt spray corrosion test duration described in step S3-2, thus obtaining the corrosion simulation results, which involve a corroded area, the morphology and type of corrosion and the size of the corrosion, for the local test assemblies of the heat exchanger.

In step S4-4, if the model credibility condition is not met, it means that the current assembly corrosion prediction model has not met the accuracy requirements, then after optimizing the parameters of the assembly corrosion prediction model (generally the optimization method is to modify the model parameters to different empirical values or change them progressively according to some rules), steps S4-3 and S4-4 are repeated until the model credibility condition is met, indicating that the current assembly corrosion prediction model has met the accuracy requirements, then the current assembly corrosion prediction model is used as the optimized assembly corrosion prediction model.

Meeting the model credibility condition means simultaneously satisfying: firstly, the corroded areas and the morphology and types of corrosion in the corrosion simulation result and the corrosion test result are the same; secondly, the error between the size of corrosion in the corrosion simulation result and corrosion test result is less than a preset corrosion size error threshold; the value range of the corrosion size error threshold is between 5% and 10%.

The specific process of step S5 includes:

In step S5-1, the air-conditioner is configured, wherein, the outdoor unit of the air-conditioner includes the air-conditioner heat exchanger to be tested, the outdoor unit is configured in the outdoor serving environment to be tested, and the indoor unit of the air-conditioner is configured indoors.

In step S5-2, a small weather station is set up in the outdoor serving environment to be tested for real-time monitoring of the atmospheric temperature, relative humidity and salt spray particle concentration in the air of the outdoor serving environment to be tested.

In step S5-3, a temperature sensor for real-time monitoring of the heat conduction pipe inlet temperature and a pressure sensor for real-time monitoring of the heat dissipation pipe inlet refrigerant pressure are configured at the heat conduction pipe inlet of the air-conditioner heat exchanger to be tested. Moreover, a wind speed sensor for real-time monitoring of the wind speed of a heat dissipation fan outlet is configured at the heat dissipation fan outlet of the air-conditioner.

In step S5-4, the air-conditioner is controlled to operate under power according to the preset verification test duration, thus conducting an outdoor verification test of the air-conditioner in the outdoor serving environment to be tested under working conditions for the air-conditioner heat exchanger to be tested. Furthermore, after the outdoor verification test of the air-conditioner is completed, based on the real-time monitoring data obtained from the small weather station, the temperature sensor, the pressure sensor and the wind speed sensor mentioned in steps S5-2 and S5-3, calculation is performed to obtain the average working condition parameters of the air-conditioner during the verification test duration. The average working condition parameters include: the average atmospheric temperature, average relative humidity and average salt spray particle concentration in the air of the outdoor serving environment to be tested, as well as the average heat conduction pipe inlet temperature, the average heat conduction pipe inlet refrigerant pressure and the average heat dissipation fan outlet wind speed of the air-conditioner.

The specific process of step S6 includes:

In step S6-1, a digitalized geometric model of the air-conditioner heat exchanger to be tested is constructed, and this digitalized geometric model of the air-conditioner heat exchanger is introduced into simulation software, wherein, the material properties of the assemblies of the digitalized geometric model of the air-conditioner heat exchanger are set as follows. The heat conduction pipe portion and the heat dissipation fin portion are set as the material of heat conduction pipe and the material of the heat dissipation fin mentioned in step S1-1 respectively, the remaining portion of the air-conditioner heat exchanger except for the heat conduction pipe and heat dissipation fin are set as steel material, and the external fluid and internal fluid of the air-conditioner heat exchanger are set as air and refrigerant respectively. The digitalized geometric model of the air-conditioner heat exchanger may be constructed through three-dimensional modeling software such as Solidworks, Auto CAD, etc.; the simulation software may be selected from simulation software such as Ansys, Comsol, etc.

In step S6-2, the average working condition parameters mentioned in step S5-4 are set as the serving environment boundary conditions of the digitalized geometric model of the air-conditioner heat exchanger, and the preset fluid and solid heat transfer models, laminar and turbulent models, and mass transfer models in the simulation software are used for solving, thereby obtaining the working condition environment field of the digitalized geometric model of the air-conditioner heat exchanger coupled with multi-physical fields of the air-conditioner heat exchanger serving environment, including: temperature field, humidity field and salt spray field.

In step S6-3, the optimized assembly corrosion prediction model mentioned in step S4 is adopted in the simulation software and the working condition environment field mentioned in step S6-2 is adopted as boundary conditions to conduct corrosion simulation calculation on the digitalized geometric model of the air-conditioner heat exchanger for any preset duration, thereby obtaining the atmospheric-corrosion prediction result of the air-conditioner heat exchanger to be tested working under working conditions in the outdoor serving environment to be tested for the preset duration. The atmospheric-corrosion prediction result includes the corroded area, the morphology and type of corrosion and size of corrosion; wherein, the preset duration may be any duration, and when the value of the verification test duration mentioned in step S5-4 is larger, the present disclosure may ensure the accuracy of the atmospheric-corrosion prediction result under a larger preset duration value.

Compared with the existing technology, the present disclosure has the following advantageous effects:

The present disclosure first conducts debugging of the material corrosion prediction model for the material of heat conduction pipe and the material of heat dissipation fin of the air-conditioner heat exchanger to be tested through steps S1 and S2, then adopts the debugged material corrosion prediction model to optimize the assembly corrosion prediction model for the local test assemblies of the heat exchanger of the air-conditioner heat exchanger to be tested through steps S3 and S4, and finally adopts the optimized assembly corrosion prediction model to achieve atmospheric-corrosion prediction for the air-conditioner heat exchanger to be tested working under working conditions in the outdoor serving environment to be tested for any preset duration through steps S5 and S6, while comprehensively simulating and coupling multiple physical fields in the air-conditioner serving environment using the working condition environment field. In this way, the atmospheric-corrosion prediction result involving the corroded area, the morphology and type of the corrosion and size of corrosion are obtained, which has the advantages of high prediction accuracy, low workload and cost.

### BRIEF DESCRIPTION OF THE DRAWINGS

The following is a further detailed explanation of the present disclosure in combination with the drawings and specific embodiments:

FIGURE is a flow diagram of the atmospheric-corrosion prediction method for air-conditioner heat exchanger of the present disclosure.

### DESCRIPTION OF THE EMBODIMENTS

The following is a detailed explanation of the present disclosure in combination with embodiments and their drawings to help those skilled in the art better understand the inventive concept of the present disclosure. However, the scope of the claims to be protected by the present disclosure is not limited to the following embodiments. For those skilled in the art, all other embodiments obtained without creative labor under the premise of not departing from the inventive concept of the present disclosure are within the scope to be protected by the present disclosure.

As shown in FIGURE, the present disclosure discloses a finite element simulation technology-based atmospheric-corrosion prediction method for air-conditioner heat exchanger, including:

In step S1, an experimental result of the corrosion rates of the material standard test specimens of a heat conduction pipe and the material standard test specimens of a heat dissipation fin is obtained through an electrochemical-corrosion experiment; wherein, the material standard test specimens of the heat conduction pipe and the material standard test specimens of the heat dissipation fin are made from the material of the heat conduction pipe and the material of the heat dissipation fin of an air-conditioner heat exchanger to be tested.

The specific process of step S1 includes:

In step S1-1, under preset experimental environmental conditions, the electrochemical-corrosion experiment is conducted on the material standard test specimens of the heat conduction pipe and the material standard test specimens of the heat dissipation fin respectively to obtain the material electrochemical data during corrosion of the material of the heat conduction pipe and the material of the heat dissipation fin; wherein, the experimental environmental conditions include temperature, relative humidity, and salt concentration. Moreover, the temperature value, relative humidity value, and salt concentration value of the experimental environmental conditions are set to the average atmospheric temperature, average relative humidity, and average salt spray particle concentration in the air calculated in step S5-4 respectively. The material electrochemical data include anodic exchange current density, anodic Tafel slope, cathode exchange current density, and cathode Tafel slope.

Generally, the material of heat conduction pipe and the material of heat dissipation fin are copper and aluminum respectively, but the present disclosure does not exclude cases where the heat conduction pipes and heat dissipation fins of the air-conditioner heat exchanger to be tested are made of other materials.

Preferably: in step S1-1, both the material standard test specimens of heat conduction pipe and the material standard test specimens of heat dissipation fin are cuboids with length, width, and thickness of 10 mm, 10 mm, and 3 mm respectively.

In step S1-2, based on the material electrochemical data obtained in step S1-1, calculation is performed to obtain the experimental result of corrosion rates of the material standard test specimens of heat conduction pipe and the material standard test specimens of heat dissipation fin under the experimental environmental conditions.

In step S2, based on the experimental parameters and result of step S1, simulation results of corrosion rates of the material standard test specimens of the heat conduction pipe and the material standard test specimens of the heat dissipation fin are obtained through simulation software; and, debugging is performed to obtain a debugged material corrosion prediction model that meets the required accuracy according to the comparison between the simulation results of the corrosion rates and the experimental result of the corrosion rates mentioned in step S1.

The specific process of step S2 includes:

In step S2-1, digitalized geometric models of the material standard test specimens of heat conduction pipe and the material standard test specimens of heat dissipation fin are constructed respectively, and the digitalized geometric models of the two material standard test specimens are introduced into simulation software; wherein, the digitalized geometric models of the material standard test specimens may be constructed through three-dimensional modeling software such as Solidworks, Auto CAD, etc.; the simulation software may be selected from simulation software with pre-set atmospheric-corrosion simulation models such as Ansys, Comsol, etc.

In step S2-2, the pre-set atmospheric-corrosion simulation model in the simulation software is adopted, and the material electrochemical data during corrosion of the material of heat conduction pipe and the material of heat dissipation fin obtained in step S1 are used as boundary conditions, so as to construct a material corrosion prediction model based on shell current distribution.

In step S2-3, in the simulation software, the material corrosion prediction model is adopted to conduct corrosion simulation calculations on the digitalized geometric models of the two material standard test specimens, thereby obtaining the simulation results of corrosion rates of the material standard test specimens of heat conduction pipe and the material standard test specimens of heat dissipation fin under the same environmental conditions as the experimental environmental conditions described in step S1-1.

In step S2-4, if the error between the simulation results of corrosion rates of the material standard test specimens of heat conduction pipe and the material standard test specimens of heat dissipation fin and the experimental result of corrosion rates obtained in step S1 is greater than the preset corrosion rate error threshold, it means that the current material corrosion prediction model has not met the accuracy requirements. Then, after debugging the parameters of the material corrosion prediction model (generally the debugging method is to modify the model parameters to different empirical values or change the model parameters progressively according to some rules), steps S2-3 and S2-4 are repeated until the error between the simulation results of corrosion rates of the material standard test specimens of heat conduction pipe and the material standard test specimens of heat dissipation fin and the experimental result of corrosion rates obtained in step S1 is less than the corrosion rate error threshold, which means that the current material corrosion prediction model has met the accuracy requirements, and the current material corrosion prediction model is then used as the debugged material corrosion prediction model. The value range of the corrosion rate error threshold is between 5% and 10%.

In step S3, salt spray corrosion tests are performed on local test assemblies of the heat

exchanger under non-working conditions to obtain the corrosion test result, which involves a corroded area, the morphology and type of corrosion and the size of the corrosion, for the local test assemblies of the heat exchanger.

The specific process of step S3 includes:

In step S3-1, a portion of the heat conduction pipes and a portion of the heat dissipation fins are cut from the air-conditioner heat exchanger to be tested through a metal cutting machine to serve as local test assemblies of the heat exchanger. Moreover, the contact method between heat conduction pipes and heat dissipation fins in the local test assemblies of the heat exchanger is consistent with the contact method between the heat conduction pipes and the heat dissipation fins in the air-conditioner heat exchanger to be tested, thus ensuring good contact between the material of heat conduction pipes and the material of heat dissipation fins.

In step S3-2, the local test assemblies of the heat exchanger are placed in a salt spray test chamber, and constant test environmental conditions are set for the salt spray test chamber. After the placement time of the local test assemblies of the heat exchanger in the salt spray test chamber reaches the preset salt spray corrosion test duration, the local test assemblies of the heat exchanger are removed for appearance observation and calibration experiment measurement, thus obtaining the corrosion test result involving the corroded area, the morphology and type of corrosion and the size of the corrosion for the local test assemblies of the heat exchanger; wherein, the test environmental conditions are the same as the experimental environmental conditions described in step S1-1. The salt concentration is realized by spraying salt solution with a mass fraction into the salt spray test chamber. The corroded area is generally divided into three types of corroded areas: corrosion occurring on heat conduction pipes, corrosion occurring on heat dissipation fins, and corrosion occurring simultaneously on heat conduction pipes and heat dissipation fins.

In step S4, based on the test parameters and result of step S3, the corrosion simulation results involving the corroded area, the morphology and type of corrosion and the size of the corrosion for the local test assemblies of the heat exchanger are obtained through simulation software; and, optimization is performed to obtain the optimized assembly corrosion prediction model that meets the required accuracy according to the comparison between the corrosion simulation results and the corrosion test result mentioned in step S3.

The specific process of step S4 includes:

In step S4-1, the digitalized geometric model of the local test assemblies of the heat

exchanger is constructed, and the digitalized geometric model of the local test assemblies is introduced into simulation software. The heat conduction pipe portion and heat dissipation fin portion in the digitalized geometric model of the local test assemblies are set with corresponding material properties, that is: the heat conduction pipe portion and heat dissipation fin portion in the digitalized geometric model of the local test assemblies are respectively set as the material of heat conduction pipe and the material of heat dissipation fin described in step S1-1 respectively. The digitalized geometric model of the local test assemblies may be constructed through three- dimensional modeling software such as Solidworks, Auto CAD, etc.; the simulation software may be selected from simulation software such as Ansys, Comsol, etc.

In step S4-2, the debugged material corrosion prediction model described in step S2 is adopted in the simulation software, and the test environmental conditions described in step S3-2 and the material electrochemical data of the material of heat conduction pipe and the material of heat dissipation fin during corrosion obtained in step SI are used as boundary conditions to construct an assembly corrosion prediction model based on shell current distribution.

In step S4-3, the assembly corrosion prediction model in the simulation software is adopted to conduct corrosion simulation calculation on the digitalized geometric model of local test assemblies according to the salt spray corrosion test duration described in step S3-2, thus obtaining the corrosion simulation results, which involve a corroded area, the morphology and type of corrosion and the size of the corrosion, for the local test assemblies of the heat exchanger.

In step S4-4, if the model credibility condition is not met, it means that the current assembly corrosion prediction model has not met the accuracy requirements, then after optimizing the parameters of the assembly corrosion prediction model (generally the optimization method is to modify the model parameters to different empirical values or change them progressively according to some rules), steps S4-3 and S4-4 are repeated until the model credibility condition is met, indicating that the current assembly corrosion prediction model has met the accuracy requirements, then the current assembly corrosion prediction model is used as the optimized assembly corrosion prediction model.

Meeting the model credibility condition means simultaneously satisfying: firstly, the corroded areas and the morphology and types of corrosion in the corrosion simulation results and the corrosion test result are the same; secondly, the error between the size of corrosion in the corrosion simulation result and corrosion test result is less than a preset corrosion size error threshold; the value range of the corrosion size error threshold is between 5% and 10%.

In step S5, an air-conditioner with an outdoor unit including the air-conditioner heat exchanger to be tested is adopted, and the outdoor unit is mounted in the outdoor serving environment to be tested so as to conduct outdoor verification tests on the air-conditioner under working conditions, thus obtaining average working condition parameters of the air-conditioner that may affect the corrosion behavior of the air-conditioner heat exchanger to be tested during the verification test duration.

The specific process of step S5 includes:

In step S5-1, the air-conditioner is configured, wherein, the outdoor unit of the air-conditioner includes the air-conditioner heat exchanger to be tested, the outdoor unit is configured in the outdoor serving environment to be tested, and the indoor unit of the air-conditioner is configured indoors.

In step S5-2, a small weather station is set up in the outdoor serving environment to be tested for real-time monitoring of the atmospheric temperature, relative humidity and salt spray particle concentration in the air of the outdoor serving environment to be tested.

In step S5-3, a temperature sensor for real-time monitoring of the heat conduction pipe

inlet temperature and a pressure sensor for real-time monitoring of the heat dissipation pipe inlet refrigerant pressure are configured at the heat conduction pipe inlet of the air-conditioner heat exchanger to be tested. Moreover, a wind speed sensor for real-time monitoring of the wind speed of a heat dissipation fan outlet is configured at the heat dissipation fan outlet of the air-conditioner.

In step S5-4, the air-conditioner is controlled to operate under power according to the preset verification test duration, thus conducting an outdoor verification test of the air-conditioner in the outdoor serving environment to be tested under working conditions for the air-conditioner heat exchanger to be tested. Furthermore, after the outdoor verification test of the air-conditioner is completed, based on the real-time monitoring data obtained from the small weather station, the temperature sensor, the pressure sensor and the wind speed sensor mentioned in steps S5-2 and S5-3, calculation is performed to obtain the average working condition parameters of the air-conditioner during the verification test duration. The average working condition parameters include: the average atmospheric temperature, average relative humidity and average salt spray particle concentration in the air of the outdoor serving environment to be tested, as well as the average heat conduction pipe inlet temperature, the average heat conduction pipe inlet refrigerant pressure and the average heat dissipation fan outlet wind speed of the air-conditioner.

In step S6, based on the average working condition parameters mentioned in step S5 and the optimized assembly corrosion prediction model mentioned in step S4, the atmospheric-corrosion prediction result for the air-conditioner heat exchanger to be tested after working for any preset duration in the outdoor serving environment to be tested under working conditions is obtained through simulation software, wherein the atmospheric-corrosion prediction result involves the corroded area, the morphology and type of corrosion and the size of the corrosion.

The specific process of step S6 includes:

In step S6-1, a digitalized geometric model of the air-conditioner heat exchanger to be tested is constructed, and this digitalized geometric model of the air-conditioner heat exchanger is introduced into simulation software, wherein, the material properties of the assemblies of the digitalized geometric model of the air-conditioner heat exchanger are set as follows. The heat conduction pipe portion and the heat dissipation fin portion are set as the material of heat conduction pipe and the material of the heat dissipation fin mentioned in step S1-1 respectively, the remaining portion of the air-conditioner heat exchanger except for the heat conduction pipe and heat dissipation fin are set as steel material, and the external fluid and internal fluid of the air-conditioner heat exchanger are set as air and refrigerant respectively. The digitalized geometric model of the air-conditioner heat exchanger may be constructed through three-dimensional modeling software such as Solidworks, Auto CAD, etc.; the simulation software may be selected from simulation software such as Ansys, Comsol, etc.

In step S6-2, the average working condition parameters mentioned in step S5-4 are set as the serving environment boundary conditions of the digitalized geometric model of the air-conditioner heat exchanger, and the preset fluid and solid heat transfer models, laminar and turbulent models, and mass transfer models in the simulation software are used for solving, thereby obtaining the working condition environment field of the digitalized geometric model of the air-conditioner heat exchanger coupled with multi-physical fields of the air-conditioner heat exchanger serving environment, including: temperature field, humidity field and salt spray field.

In step S6-3, the optimized assembly corrosion prediction model mentioned in step S4 is adopted in the simulation software and the working condition environment field mentioned in step S6-2 is adopted as boundary conditions to conduct corrosion simulation calculation on the digitalized geometric model of the air-conditioner heat exchanger for any preset duration, thereby obtaining the atmospheric-corrosion prediction result of the air-conditioner heat exchanger to be tested working under working conditions in the outdoor serving environment to be tested for the preset duration. The atmospheric-corrosion prediction result includes the corroded area, the morphology and type of corrosion and size of corrosion; wherein, the preset duration may be any duration, and when the value of the verification test duration mentioned in step S5-4 is larger, the present disclosure may ensure the accuracy of the atmospheric-corrosion prediction result under a larger preset duration value.

In this way, the present disclosure first conducts debugging of the material corrosion prediction model for the material of heat conduction pipe and the material of heat dissipation fin of the air-conditioner heat exchanger to be tested through steps S1 and S2, then adopts the debugged material corrosion prediction model to optimize the assembly corrosion prediction model for the local test assemblies of the heat exchanger of the air-conditioner heat exchanger to be tested through steps S3 and S4, and finally adopts the optimized assembly corrosion prediction model to achieve atmospheric-corrosion prediction for the air-conditioner heat exchanger to be tested working under working conditions in the outdoor serving environment to be tested for any preset duration through steps S5 and S6, while comprehensively simulating and coupling multiple physical fields in the air-conditioner serving environment using the working condition environment field. Accordingly, the atmospheric-corrosion prediction result involving the corroded area, the morphology and type of the corrosion and size of corrosion are obtained, which has the advantages of high prediction accuracy, low workload and cost.

The following are the parameter settings and process data in an example of the present disclosure:

In step S1-1, the experimental environmental conditions are set as: temperature at 25° C., relative humidity at 85%, salt concentration at standard 5% salt solution.

In step S1-1, the material electrochemical data include: the anodic exchange current density, anodic Tafel slope, cathodic exchange current density, and cathodic Tafel slope of the material of heat conduction pipe are 0.0001 A/m², 0.1V, 0.001 A/m², and −0.1V, respectively; the anodic exchange current density, anodic Tafel slope, cathodic exchange current density, and cathodic Tafel slope of the material of heat dissipation fin are 0.0001 A/m², 0.1V, 0.000001 A/m², and −0.1V, respectively.

In step S1-2, the experiment result of corrosion rates of the material standard test specimen of heat conduction pipe and material standard test specimen of heat dissipation fin are: 631.8 um/a and 108.7 um/a, respectively.

In step S2-3, the simulation results of corrosion rates of the material standard test specimen of heat conduction pipe and the material standard test specimen of heat dissipation fin are: 611.3 um/a and 105.1 um/a, respectively.

In step S2-4, the corrosion rate error threshold is set at 5%. After debugging the parameters of the material corrosion prediction model, the error between the simulation results of corrosion rates of the material standard test specimen of heat conduction pipe and the material standard test specimen of heat dissipation fin and the experiment result of corrosion rates obtained in step S1 are: 3.2% and 3.3%, respectively, both below the corrosion rate error threshold, indicating that the current material corrosion prediction model has met the accuracy requirements. In step S3-2, the test environmental conditions are: temperature at 25° C., relative

humidity at 85%, salt concentration at 5% mass fraction salt solution; the salt spray corrosion test duration is 24 hours; the corrosion test results are: the corroded area occurs on the heat dissipation fin, morphology and type of corrosion, and size of corrosion is 0.433 um.

In step S4-3, the corrosion simulation results are: the corroded area occurs on the heat dissipation fin, the morphology and type of corrosion is the same as that in step S3-2, and the size of corrosion is 0.452 um.

In step S4-4, the corrosion amount error threshold is set at 5%. After optimizing the parameters of the assembly corrosion prediction model, the corroded area and morphology and type of corrosion in the corrosion simulation results and corrosion test results are the same, and the error between the size of corrosion of the corrosion simulation results and corrosion test results is 4.4%, which is below the corrosion amount error threshold, indicating that the current assembly corrosion prediction model has met the accuracy requirements.

In step S5-4, the verification test duration is 1 year, and the measured average working condition parameters are: average atmospheric temperature is 27.6° C., average relative humidity is 86%, average salt spray particle concentration in the air is 1.2 mg/m³, average heat pipe inlet temperature is 75° C., average heat pipe inlet refrigerant pressure is 1.3 MPa, and average wind speed at the heat dissipation fan outlet is 3 m/s.

In step S6, the present disclosure may accurately predict the corrosion prediction results of the tested air-conditioner heat exchanger, and the obtained atmospheric-corrosion prediction results are: the corroded area is on the heat dissipation fin, the morphology and type of corrosion is local corrosion, and the size of corrosion is 3.44 um.

The present disclosure is not limited to the specific embodiments described above. Based on the above content, according to common technical knowledge and conventional methods in this field, the present disclosure may also be subject to various forms of equivalent modifications, substitutions or alterations without departing from the basic technical concept of the present disclosure described above, and all of which fall within the scope to be protected by the present disclosure.

What is claimed is:

1. A finite element simulation technology-based atmospheric-corrosion prediction method for an air-conditioner heat exchanger, comprising:
step S1, an experimental result of corrosion rates of a material standard test specimens of a heat conduction pipe and a material standard test specimens of a heat dissipation fin is obtained through an electrochemical-corrosion experiment; wherein, the material standard test specimens of the heat conduction pipe and the material standard test specimens of the heat dissipation fin are made from a material of the heat conduction pipe and a material of the heat dissipation fin of an air-conditioner heat exchanger to be tested;
a specific process of the step S1 comprising:
step S1-1, under preset experimental environmental conditions, the electrochemical-corrosion experiment is conducted on the material standard test specimens of the heat conduction pipe and the material standard test specimens of the heat dissipation fin respectively to obtain material electrochemical data during corrosion of the material of the heat conduction pipe and the material of the heat dissipation fin; wherein, the preset experimental environmental conditions comprise temperature, relative humidity, and salt concentration, the material electrochemical data comprise an anodic exchange current density, an anodic Tafel slope, a cathode exchange current density, and a cathode Tafel slope;
step S1-2, based on the material electrochemical data obtained in step S1-1, calculation is performed to obtain the experimental result of the corrosion rates of the material standard test specimens of the heat conduction pipe and the material standard test specimens of the heat dissipation fin under the experimental environmental conditions;
step S2, based on experimental parameters and a result of step S1, simulation results of the corrosion rates of the material standard test specimens of the heat conduction pipe and the material standard test specimens of the heat dissipation fin are obtained through simulation software; and, debugging is performed to obtain a debugged material corrosion prediction model according to a comparison between the simulation results of the corrosion rates and the experimental result of the corrosion rates mentioned in step S1;
step S3, salt spray corrosion tests are performed on local test assemblies of a heat exchanger under non-working conditions to obtain a corrosion test result, which involves a corroded area, a morphology and type of the corrosion and a size of the corrosion, for the local test assemblies of the heat exchanger;
step S4, based on test parameters and a result of step S3, corrosion simulation results involving the corroded area, the morphology and type of the corrosion and the size of the corrosion for the local test assemblies of the heat exchanger are obtained through the simulation software; and, optimization is performed to obtain an optimized assembly corrosion prediction model according to a comparison between the corrosion simulation results and the corrosion test result mentioned in step S3;
step S5, an air-conditioner with an outdoor unit comprising the air-conditioner heat exchanger to be tested is adopted, and the outdoor unit is mounted in an outdoor serving environment to be tested so as to conduct outdoor verification tests on the air-conditioner under working conditions, thus obtaining average working condition parameters of the air-conditioner during a verification test duration;
step S6, based on the average working condition parameters mentioned in step S5 and the optimized assembly corrosion prediction model mentioned in step S4, an atmospheric-corrosion prediction result for the air-conditioner heat exchanger to be tested after working for any preset duration in the outdoor serving environment to be tested under the working conditions is obtained through the simulation software, wherein the atmospheric-corrosion prediction result involves the corroded area, the morphology and type of the corrosion and the size of the corrosion;
a specific process of the step S5 comprising:
step S5-1, the air-conditioner is configured, wherein, the outdoor unit of the air-conditioner comprises the air-conditioner heat exchanger to be tested, the outdoor unit is configured in the outdoor serving environment to be tested, and an indoor unit of the air-conditioner is configured indoors;
step S5-2, a small weather station is set up in the outdoor serving environment to be tested for real-time monitoring of an atmospheric temperature, a relative humidity and a salt spray particle concentration in the air of the outdoor serving environment to be tested;
step S5-3, a temperature sensor for real-time monitoring of a heat conduction pipe inlet temperature and a pressure sensor for real-time monitoring of a heat dissipation pipe inlet refrigerant pressure are configured at a heat conduction pipe inlet of the air-conditioner heat exchanger to be tested, a wind speed sensor for real-time monitoring of a wind speed of a heat dissipation fan outlet is configured at the heat dissipation fan outlet of the air-conditioner;
step S5-4, the air-conditioner is controlled to operate under power according to a preset verification test duration, thus conducting the outdoor verification tests on the air-conditioner in the outdoor serving environment to be tested under the working conditions for the air-conditioner heat exchanger to be tested; after the outdoor verification tests on the air-conditioner is completed, based on real-time monitoring data obtained from the small weather station, the temperature sensor, the pressure sensor and the wind speed sensor mentioned in steps S5-2 and S5-3, calculation is performed to obtain the average working condition parameters of the air-conditioner during the verification test duration, the average working condition parameters comprise: an average atmospheric temperature, an average relative humidity and an average salt spray particle concentration in the air of the outdoor serving environment to be tested, as well as an average heat conduction pipe inlet temperature, an average heat conduction pipe inlet refrigerant pressure and an average heat dissipation fan outlet wind speed of the air-conditioner;
a specific process of the step S6 comprising:
step S6-1, a digitalized geometric model of the air-conditioner heat exchanger to be tested is constructed, and the digitalized geometric model of the air-conditioner heat exchanger is introduced into the simulation software, wherein, material properties of assemblies of the digitalized geometric model of the air-conditioner heat exchanger are set as follows: a heat conduction pipe portion and a heat dissipation fin portion are set as the material of the heat conduction pipe and the material of the heat dissipation fin mentioned in step S1-1 respectively, a remaining portion of the air-conditioner heat exchanger except for the heat conduction pipe and the heat dissipation fin are set as steel material, and an external fluid and an internal fluid of the air-conditioner heat exchanger are set as air and refrigerant respectively;
step S6-2, the average working condition parameters mentioned in step S5-4 are set as serving environment boundary conditions of the digitalized geometric model of the air-conditioner heat exchanger, and fluid and solid heat transfer models, laminar and turbulent models, and mass transfer models in the simulation software are used for solving, thereby obtaining a working condition environment field of the digitalized geometric model of the air-conditioner heat exchanger, comprising: a temperature field, a humidity field and a salt spray field;
step S6-3, the optimized assembly corrosion prediction model mentioned in step S4 is adopted in the simulation software and the working condition environment field mentioned in step S6-2 is adopted as boundary conditions to conduct a corrosion simulation calculation on the digitalized geometric model of the air-conditioner heat exchanger for the any preset duration, thereby obtaining the atmospheric-corrosion prediction result of the air-conditioner heat exchanger to be tested working under the working conditions in the outdoor serving environment to be tested for the preset duration, the atmospheric-corrosion prediction result comprises the corroded area, the morphology and type of the corrosion and size of corrosion.

2. The finite element simulation technology-based atmospheric-corrosion prediction method for the air-conditioner heat exchanger according to claim 1, wherein in the step S1-1, both the material standard test specimens of the heat conduction pipe and the material standard test specimens of the heat dissipation fin are cuboids with length, width, and thickness of 10 mm, 10 mm, and 3 mm respectively.

3. The finite element simulation technology-based atmospheric-corrosion prediction method for the air-conditioner heat exchanger according to claim 1, wherein a specific process of the step S2 comprises:
step S2-1, digitalized geometric models of the material standard test specimens of the heat conduction pipe and the material standard test specimens of the heat dissipation fin are constructed respectively, and the digitalized geometric models of the two material standard test specimens are introduced into the simulation software;
step S2-2, an atmospheric-corrosion simulation model in the simulation software is adopted, and the material electrochemical data during the corrosion of the material of the heat conduction pipe and the material of the heat dissipation fin obtained in step S1 are used as the boundary conditions, so as to construct a material corrosion prediction model based on shell current distribution;
step S2-3, in the simulation software, the material corrosion prediction model is adopted to conduct corrosion simulation calculations on the digitalized geometric models of the two material standard test specimens, thereby obtaining the simulation results of the corrosion rates of the material standard test specimens of the heat conduction pipe and the material standard test specimens of the heat dissipation fin under same environmental conditions as the experimental environmental conditions described in step S1-1;
step S2-4, if an error between the simulation results of the corrosion rates of the material standard test specimens of the heat conduction pipe and the material standard test specimens of the heat dissipation fin and the experimental result of the corrosion rates obtained in step S1 is greater than a preset corrosion rate error threshold, after debugging parameters of the material corrosion prediction model, steps S2-3 and S2-4 are repeated until the error between the simulation results of the corrosion rates of the material standard test specimens of the heat conduction pipe and the material standard test specimens of the heat dissipation fin and the experimental result of the corrosion rates obtained in step S1 is less than the corrosion rate error threshold, and the current material corrosion prediction model is then used as the debugged material corrosion prediction model.

4. The finite element simulation technology-based atmospheric-corrosion prediction method for the air-conditioner heat exchanger according to claim 1, wherein a specific process of the step S3 comprises:
step S3-1, a portion of the heat conduction pipes and a portion of the heat dissipation fins are cut from the air-conditioner heat exchanger to be tested to serve as the local test assemblies of the heat exchanger, and a contact method between the heat conduction pipes and the heat dissipation fins in the local test assemblies of the heat exchanger is consistent with a contact method between the heat conduction pipes and the heat dissipation fins in the air-conditioner heat exchanger to be tested;
step S3-2, the local test assemblies of the heat exchanger are placed in a salt spray test chamber, and constant test environmental conditions are set for the salt spray test chamber, after a placement time of the local test assemblies of the heat exchanger in the salt spray test chamber reaches a preset salt spray corrosion test duration, the local test assemblies of the heat exchanger are removed for appearance observation and calibration experiment measurement, thus obtaining the corrosion test result involving the corroded area, the morphology and type of the corrosion and the size of the corrosion for the local test assemblies of the heat exchanger; wherein, the test environmental conditions are the same as the experimental environmental conditions described in step S1-1.

5. The finite element simulation technology-based atmospheric-corrosion prediction method for the air-conditioner heat exchanger according to claim 4, wherein a specific process of the step S4 comprises:
step S4-1, a digitalized geometric model of the local test assemblies of the heat exchanger is constructed, and the digitalized geometric model of the local test assemblies is introduced into the simulation software, wherein the heat conduction pipe portion and the heat dissipation fin portion in the digitalized geometric model of the local test assemblies are set with corresponding material properties;
step S4-2, the debugged material corrosion prediction model described in step S2 is adopted in the simulation software, and the test environmental conditions described in step S3-2 and the material electrochemical data of the material of the heat conduction pipe and the material of the heat dissipation fin during the corrosion obtained in step S1 are used as the boundary conditions to construct the assembly corrosion prediction model based on the shell current distribution;
step S4-3, the assembly corrosion prediction model in the simulation software is adopted to conduct the corrosion simulation calculation on the digitalized geometric model of the local test assemblies according to the salt spray corrosion test duration described in step S3-2, thus obtaining the corrosion simulation results, which involve the corroded area, the morphology and type of the corrosion and the size of the corrosion, for the local test assemblies of the heat exchanger;
step S4-4, if a model credibility condition is not met, then after optimizing parameters of the assembly corrosion prediction model, steps S4-3 and S4-4 are repeated until the model credibility condition is met, then the current assembly corrosion prediction model is used as the optimized assembly corrosion prediction model;
wherein meeting the model credibility condition means simultaneously satisfying: firstly, the corroded areas and the morphology and types of the corrosion in the corrosion simulation results and the corrosion test result are the same; secondly, the error between the size of the corrosion in the corrosion simulation results and the corrosion test result is less than a preset corrosion size error threshold.
